SEQUENCE LISTING (2 sequences)

SEQ ID NO: 1 - protein, 520 aa, Haemophilus parasuis
MKPYLSKIVIATLFSSSVQVVEAQTYWASGVNQNSGWTADLQYPNQCWGAVAGNTLGWWKSRVKAQVEFNEDTPKDSKAISGWIYKTYPHIQGGLQPYRGMEYFFSRFASGVKLYEEHNKKTYYESERGPTKISGGPFWTERYDSDAKLVTKSLIDNFKTGNVVAALTSQHHTVTLWGIEVDGDGKIKKGWISDSVKDKAGNLKMVEVVGNYAKDNKGNDIFRFLYSYAVDGGRMYVTDLYDIYSITYLSIDEARNNGTYKDTSNREDCRLSLAPGVSESFCSINSTATNTTKVENVSTTSNNSTNLPTKVENSGHSNQAINAASASDDTVSSASENNDSVNTPENIENSNGVDIGNNSTVSSANVENSSNEVSVINTPNTSSLPNDQGTKKIIVDSSESNAKDPIISKATESLTKEINSYPELSFITKNMGDVTFYIVQKDGVDITLINPKTEQSLVSNKGLVIYDFKQGQNGNIHITKSPNQAAIKEANEVTSLHTDLNHVEMNNLNKRMGELRGIDA

SEQ ID NO: 2 - protein, 92 aa, Haemophilus parasuis
PTKISGGPFWTERYDSDAKLVTKSLIDNFKTGNVVAALTSQHHTVTLWGIEVDGDGKIKKGWISDSVKDKAGNLKMVEVVGNYAKDNKGNDI

(19) United States
(12) Patent Application Publication
(10) Pub. No.: US 2022/0378900 A1
(43) Pub. Date: Dec. 1, 2022

(54) A NOVEL VACCINE AGAINST HEAMOPHILUS PARASUIS

(71) Applicant: Intervet Inc., Madison, NJ (US)

(72) Inventors: Antonius Arnoldus Christiaan Jacobs, Kessel (NL); Theodora Johanna Van Kasteren-Westerneng, Arnhem (NL); Johanna Jacoba Elisabeth Bijlsma, Nijmegen (NL)

(73) Assignee: Intervet Inc., Madison, NJ (US)

(21) Appl. No.: 17/775,428

(22) PCT Filed: Nov. 19, 2020
(86) PCT No.: PCT/EP2020/082634

§ 371 Date: May 9, 2022

(30) Foreign Application Priority Data
Nov. 20, 2019 (EP) 19210262.2

(51) Int. Cl. A61K 39/102; A61P 31/04

(52) U.S. Cl. CPC A61K 39/102; A61P 31/04

(57) ABSTRACT

The invention pertains to a protein having at least 69% sequence identity with the protein according to SEQ ID No: 1 or an immunogenic fragment of this protein, for use in a prophylactic method to protect a pig against an infection with Haemophilus parasuis by administering a vaccine to the pig, the vaccine comprising the protein or the immunogenic fragment thereof as an antigen. The invention also pertains to a vaccine, a method to manufacture such a vaccine and a method to protect a pig against H. parasuis.

### GENERAL FIELD OF THE INVENTION

The invention in general pertains to the treatment of pigs against an infection with the pathogenic bacterium Haemohilus parasuis. In particular the invention pertains to a novel vaccine for prophylactically treating pigs against an infection with this bacterium.

### BACKGROUND OF THE INVENTION

Haemophilus parasuis is one of the most important bacteria affecting pigs. The disease caused by this pathogen is characterized by polyserositis and it is known as Glasser's disease. Haemophilus parasuis is present in all major swine-rearing countries and remains a significant pathogen in contemporary swine production systems. In addition to causing disease, Haemophilus parasuis is frequently isolated from the upper respiratory tract of healthy pigs. There are different serotypes known of Haemophilus parasuis, each of these can be identified using the technique of immunodiffusion (Kielstein et al. in J. Clin. Microbiol. 30:862-865; 1992 and Rapp-Gabrielson et al. in AJVR 53:659-664; 1992). Successful vaccination resulting in decreased mortality has been achieved by various types of vaccines.

Inactivated H. parasuis (i.e. bacterin) vaccines are widely used today. All commercially available H. parasuis vaccines are inactivated vaccines. Most of the currently available commercial vaccines are produced by propagating a virulent H. parasuis strain where after the strain is inactivated. Bacterial cultures are pelleted by high-speed centrifugation and resuspended in sterile phosphate buffer saline, and subsequently formulated with an appropriate adjuvant such as mineral oil, aluminum hydroxide, Carbopol, saponin, vitamin E acetate, squalene, squalene etc. In addition to monovalent vaccines, there are bivalent, trivalent, or tetravalent H. parasuis vaccines, which include various serotypes. They generally provide a low level of cross-protection, and they are more efficacious against homologous serotypes. These inactivated vaccines, such as for example Porcilis Glasser (MSD, Boxmeer, The Netherlands) play important roles in controlling Glasser's disease outbreaks throughout the world.

Stably attenuated H. parasuis strains could in theory serve as safe and efficacious vaccines. However, the development of attenuated H. parasuis vaccines has been limited because of the lack of knowledge regarding the major virulence factors of H. parasuis, which makes it difficult to create H. parasuis mutants that could serve as potential vaccines. To date, there are no genetically engineered, live attenuated or inactivated H. parasuis vaccine candidates.

Several subunit vaccines have been studied, but existing data suggest that a few subunit vaccines induce high levels of anti-H. parasuis neutralizing antibodies and protect pigs against H. parasuis challenge. However, commercial subunit vaccines that prevent and control Glässer's disease are not available currently. Recently (Huisheng Liu et al, in Veterinary Immunology and Immunopathology, Volume 180, 1 Nov. 2016, pp 53-58), subunit vaccines that comprise newly identified protective antigens such as recombinant transferrin-binding protein B (TbpB), outer membrane protein (OMP) formulations enriched with TbpB, OMP2 and OMP5, transferrin-binding protein A (TbpA), trimeric autotransporters (VtaA), six secreted proteins (PflA, Gcp, Ndk, HsdS, RnfC, and HAPS_0017), three glyceraldehyde-3-phosphate dehydrogenase (GAPDH), OapA, and HPS-0675 fusion proteins, various alternative OMPs (SmpA, YgiW, and FOG), 6-phosphogluconate-dehydrogenase, cytolethal distending toxin subunits A, B, and C, and neuraminidase or lipoprotein, have been proven to provide partial protection against H. parasuis challenge.

To date, also a DNA vaccine has been shown to be able and provide some partial protection against H. parasuis. The vaccine comprises DNA encoding H. parasuis GAPDH.

However despite the commercial availability of vaccines, antimicrobials still are widely used to treat H. parasuis infections mainly due to incomplete efficacy of many existing vaccines. Pigs receiving antimicrobials early during infection with H. parasuis are usually able to survive a systemic infection. However, there is a lot of pressure to reduce the amount of antimicrobials used for growing pigs.

### OBJECT OF THE INVENTION

It is an object to provide an alternative vaccine to prophylactically treat a pig against an infection with H. parasuis, which vaccine preferably provides protection at least as good as a conventional bacterin vaccine.

### SUMMARY OF THE INVENTION

In order to meet the object of the invention, it was found that a protein having at least 69% sequence identity with the protein according to SEQ ID No: 1 or an immunogenic fragment of this protein, can be used in a prophylactic method to protect a pig against an infection with Haemophilus parasuis by administering a vaccine to the pig, the vaccine comprising the protein or the immunogenic fragment thereof as an antigen.

The fact that this protein or an immunogenic fragment thereof could be used in order to treat a pig against an infection with H. parasuis was based on the surprising finding that the native protein, a putative serine protease which is conserved in various H. parasuis serotypes (including the virulent serotypes 4, 5, 12, 13 and 15), plays a key role in the infection with H. parasuis. This could be established since vaccinating with (part of) the naturally occurring protein led to a very good protection against pathogenic H. parasuis, at a level that is even better than protection that can be arrived at a conventional and well established bacterin vaccine. This shows that this serine protease plays a key role in the pathogenicity of the bacterium, and that neutralizing the function of this protein helps in decreasing the infection, including the clinical disease resulting therefrom. In that respect, the merit of the inventors lies in the recognition that this serine protease plays a key role in the pathogenicity of H. parasuis. Once this was recognized, it followed that inducing antibodies against this protein would be effective as a treatment against an infection with H. parasuis. The most straightforward way of inducing such antibodies is to administer a protein or polypeptide that resembles the wild type protein.

With respect to the protein as such, across various serotypes, the natural variation of the protein over its full length is about 69% with respect to the protein having an amino acid sequence according to SEQ ID No:1. So using a protein that meets this identity level can be used to arrive at protection against the various wild type H. parasuis strains of different serotypes that naturally produce the corresponding protein.

Although for finding the gist of the invention the protein according to the (complete) SEQ ID No:1 was used as antigen to induce antibodies against the natural protein, it is commonly known that when antibodies need to be raised against a certain (naturally occurring) protein, it is typically not necessary to use the whole protein. It is also possible to use an immunogenic fragment of that protein that is capable, as such or coupled to a carrier such as e.g. KLH, of inducing an immune response against the corresponding protein. This is in particular true for the serine protease of the present invention. To start with, the protein according to SEQ ID No:1 is already only a part of the naturally occurring protein, which includes an autotransporter beta barrel. Next to this, although its function in the pathogenicity of H. parasuis remains unknown, it was found that the protein is homologous to the human Mac-1 protein. This could be established via a so called Cobalt alignment: a multiple sequence alignment tool that finds a collection of pairwise constraints derived from conserved domain database, protein motif database, and sequence similarity, using RPS-BLAST, BLASTP, and PHI-BLAST, wherein pairwise constraints are then incorporated into a progressive multiple alignment (see Papadopoulos J S and Agarwala R, Bioinformatics 23:1073-79, 2007; PMID: 17332019). This was confirmed by a search in the NCBI database for “Mac-1 family” protein in “haemophilus” bacteria, which confirmed the presence of a protein with a Mac-1 domain homologue in H. parasuis. The Mac-1 protein on its turn is known to be homologous to an IgM protease of the pig pathogenic bacterium Streptococus suis as described in WO 2015/181356 (IDT Biologika GmbH). As taught in WO 2015/181356, a vaccine directed against the full length protein protein or a fragment comprising only the highly conserved Mac-1 domain, is able to induce antibodies against the full length naturally occurring protein, therewith providing protection against the corresponding bacterium. Although H. parasuis is totally unrelated to S. suis, the fact that both bacteria produce a protein with a Mac-1 domain homologue, which is known to have immunoglobulin peptidase activity (see PFAM database of the European Molecular Biology Institute, as confirmed in WO 2015/181356), combined with the fact that the diseases are clinically alike (they both lead to polyserositis, typically are pathogenic only for piglets, and are both induced by stress such as weaning and transport), indicates that also in H. parasuis, like in Streptococcus suis, the corresponding natural protein is involved in immune evasion. Moreover, the fact that in both pathogenic bacteria the Mac-1 domain is highly conserved across serotypes, but that for the remainder the H. parasuis and Streptococcus suis proteins are completely unrelated (identity level over the whole protein, inluding the Mac-1 domain, is as low as 13%), combined with the fact that across H. parasuis serotypes 3, 4, 5, 9, 12, 13 and 15 the identity of the Mac-1 domain is even 100% (whereas for the remainder of the protein this is much lower, down to 69%), even evidence that the Mac-1 domain contains the prime immunogenic epitope(s), and thus on itself is able to induce antibodies that neutralise the naturally occurring serine protease that comprises this so-called Mac-1 domain. Preferably the fragment comprises the naturally occurring Mac-1 domain of H. parasuis, i.e. the sequence according to SEQ ID NO:2. However, as is commonly known, small variations of 10-20%, even up to 30% in sequence identity, may still be useful as effective antigen and capable of inducing neutralising antibodies. These variations can be reflected by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science 227, 1435-1441, 1985) and determining the functional similarity between homologous proteins. Such amino acid substitutions of the exemplary embodiments of this invention, as well as variations having deletions and/or insertions are within the scope of the invention. Therefore, the fragment for use in the present invention should comprise a polypeptide that is at least 70% identical to the polypeptide according to SEQ ID NO:2, preferably at least 75, 80, 85, 90, 91, 92, 93, 94, 95, 96, 97, 98, 99% or higher.

All in all, it was found that using the protein according to the invention in a vaccine, one is able to induce antibodies that are directed against the naturally occurring H. parasuis serine protease. This means that one is able to treat a (post-vaccination) infection with H. parasuis. Next to this, based on homology with the human Mac-1 protein and Streptococcus suis which produces an IgM protease comprising a Mac-1 domain, it is understood that a polypeptide comprising (at least) the Mac-1 domain of the current serine protease (optionally coupled to an immunogenic carrier such as for example KLH) is sufficient, when used as antigen in a vaccine, to induce antibodies against the naturally occurring protein.

The invention is also embodied in a vaccine to protect a pig against an infection with Haemophilus parasuis, the vaccine comprising a protein having at least 69% sequence identity with the protein according to SEQ ID No: 1 or an immunogenic fragment of this protein, and a pharmaceutically acceptable carrier.

The invention also pertains to the use of a protein having at least 69% sequence identity with the protein according to SEQ ID No: 1 or an immunogenic fragment of this protein, as an antigen for manufacturing a vaccine for protecting a pig against an infection with Haemophilus parasuis.

Next to this, the invention pertains to a method to protect a pig against an infection with Haemophilus parasuis by administering a vaccine to the pig, the vaccine comprising as an antigen a protein having at least 69% sequence identity with the protein according to SEQ ID No: 1 or an immunogenic fragment of this protein.

Definitions

An antigen is antigenic material derived from a micro-organism, notwithstanding that the antigen is ultimately artificially produced. An antigen initiates and mediates the formation of an antibody that acts against the corresponding naturally occurring compound (typically a protein). Bacteria, viruses, protozoans, and other microorganisms are important sources of antigens. These may for example be proteins or polysaccharides derived from the outer surfaces of the cell (capsular antigens), from the cell interior (the somatic or O antigens), from the flagella (the flagellar or H antigens), or from excreted products including for example enzymes and toxins.

A vaccine is a constitution suitable for application to an animal, comprising one or more antigens in an immunologically effective amount, i.e. capable of stimulating the immune system of the target animal sufficiently to induce an immune response, such as antibodies, against the antigens and therewith against the corresponding naturally occurring proteins, typically combined with a pharmaceutically acceptable carrier (i.e. a biocompatible medium, viz. a medium that after administration does not induce significant adverse reactions in the subject animal, capable of presenting the antigen to the immune system of the host animal after administration of the vaccine) such as a liquid containing water and/or any other biocompatible solvent or a solid carrier such as commonly used to obtain freeze-dried vaccines (based on sugars and/or proteins), optionally comprising immunostimulating agents (adjuvants), which upon administration to the animal induces an immune response that is able to protect the animals against a (post-vaccinating) infection.

A prophylactic method is a method designed to protect against an infection or the corresponding disease by acting before the infection actually occurs, typically by treating a subject animal with a vaccine before the subject animal is expected to become infected.

To protect a pig against an infection with H. parasuis means aiding in preventing, ameliorating or curing a pathogenic infection with H. parasuis, or aiding in preventing, ameliorating or curing a disorder arising from that infection, for example to prevent or reduce one or more clinical signs resulting from the infection with H. parasuis.

An immunogenic fragment is a fragment of a protein that still has retained its capability to induce an immune response in a host, i.e. comprises a B- or T-cell epitope. A variety of techniques is commonly available to easily identify immunogenic fragments (determinants), in particular immunogenic fragments of proteins. The method described by Geysen et al (Patent Application WO 84/03564, Patent Application WO 86/06487, U.S. Pat. No. 4,833,092, Proc. Natl Acad. Sci. 81: 3998-4002 (1984), J. Imm. Meth. 102, 259-274 (1987), the so-called PEPSCAN method is an easy to perform, quick and well-established method for the detection of immunogenic epitopes of proteins. The method is used world-wide and as such well-known to man skilled in the art. This (empirical) method is especially suitable for the detection of B-cell epitopes. Also, given the sequence of the gene encoding any protein, computer algorithms are able to designate specific protein fragments as the immunologically important epitopes on the basis of their sequential and/or structural agreement with epitopes that are now known. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235, 1059-1062 (1987) and US Patent application NTIS U.S. Ser. No. 07/005,885). A condensed overview is found in: Shan Lu on common principles: Tibtech 9: 238-242 (1991), Good et al on Malaria epitopes; Science 235: 1059-1062 (1987), Lu for a review; Vaccine 10: 3-7 (1992), Berzofsky for HIV-epitopes; The FASEB Journal 5:2412-2418 (1991). It is common general knowledge that peptides in order to be immunogenic need to be of a minimal length; 8-11 aa for MHC I receptor binding, and 11-15 aa for MHC II receptor binding (reviewed e.g. by R. N. Germain & D. H. Margulies, 1993, Annu. Rev. Immunol., vol. 11, p. 403-450: The biochemistry and cell biology of antigen processing and presentation).

Sequence identity between two polypeptides (or nucleic acids) means the percentage of identical amino acids (or nucleotides) in overlapping regions of the polypeptides (or nucleic acids) as established with the BLAST program using the blastp algorithm with default parameters (see Tatiana A. Tatusova, Thomas L. Madden FEMS Microbiol. Letters 174: 247-250; 1999).

Further Embodiments of the Invention

In a further embodiment the protein has at least 90% sequence identity with the protein according to SEQ ID No: 1, even at least 95% sequence identity with the protein according to SEQ ID No: 1, up to 100% sequence identity.

In another embodiment the protein or immunogenic fragment are used in a method to protect the pig against an increased risk of mortality due to the infection with Haemophilus parasuis.

In yet another embodiment the protein or immunogenic fragment are used in a method to protect the pig against one or more clinical signs due to the infection with Haemophilus parasuis.

The invention will now be further illustrated using the following specific examples.

### EXAMPLES

### Example 1

Objective

The objective of this alignment experiment was to find the sequence identity level of the serine protease across various H. parasuis strains of various serotypes, and to identify the Mac-1 domain in the serine protease.

Results

In the table here below, it is indicated what the sequence identity is with respect to SEQ ID No:1 for the corresponding serine proteases in other H. parasuis strains. It appears that the level of identity is at least 69% among various strains of common serotypes. For strains within the group of known highly pathogenic and most prevalent serotypes 4, 5, 12 and 13, the level of identity is even 90% or higher.

TABLE 1
Sequence identity with SEQ ID No: 1 for various H. parasuis strains

| Strain | Start | End | % Identity |
|---|---|---|---|
| Serotype 3-strain SW1 | 1 | 491 | 69 |
| Serotype 4-strain GX0 | 1 | 471 | 90 |
| Serotype 4-strain HPS | 1 | 523 | 97 |
| Serotype 5-strain 297 | 1 | 523 | 99 |
| Serotype 5-strain Nag | 1 | 523 | 98 |
| Serotype 5-strain SH0 | 1 | 523 | 99 |
| Serotype 9-strain D74 | 1 | 727 | 70 |
| Serotype 12-strain ZJ | 1 | 523 | 98 |
| Serotype 13-strain MN | 1 | 503 | 95 |
| Serotype 15-strain 84 | 1 | 523 | 99 |

Next to the above, the Mac-1 domain of H. parasuis was identified, and herewith disclosed as SEQ ID No:2. The protein according to SEQ ID No:1 is derived from the putative extracellular serine protease of Haemophilus parasuis serotype 5, strain SH0165 (Genbank No ACL32961.1), having a length of 780 amino acids. When removing the autotransporter domain (AA's 521 to 780), and performing a HMMR identification (see www.hmmer.org; Robert Finn et al in Nucleic Acids Research, 2011 Jul 1; 39, Web Server issue, W29—W37), the Mac-1 family domain is indicated to start at AA 130 and ending at AA 221.

When comparing the Mac-1 domain of H. parasuis with the Mac-1 domain of Streptococcus suis there is only a 17% identity (using Blastp), which is an indication that only a small part of the domain is essential for the antibody binding and/or protease activity. Next to this, this is an indication that although the Mac-1 domain is present with 100% identity in various H. parasuis serotypes (as indicated here above; notwithstanding that in other strains or serotypes the level of identity is lower), variations of the naturally occurring protein are likely to induce effective antibodies against the natural protein, at least variations within 70% identity level or more.

### Example 2

Objective

The objective of this study was to test the efficacy of a subunit vaccine compared to a conventional bacterin vaccine against H. parasuis serotype 5 challenge. The subunit vaccine comprised the polypeptide according to SEQ ID No:1, wherein the corresponding DNA was cloned from H. parasuis serotype 5, strain SH0165 (Genbank no. ACL32961.1), expressed in an E. coli expression vector system (pET22b, with pelB signal sequence and a HIS tag). The bacterin vaccine contained inactivated cells of Haemophilus parasuis bacteria of serotype 5.

Study Design

For this study thirty healthy piglets at 4 weeks of age were used. The piglets were allotted to three groups (evenly distributed over the different litters) of 10 piglets each.

Group 1 was vaccinated twice intramuscularly at 4 and 6 weeks of age with 2 ml of a vaccine containing the subunit at 75 μg/ml, suspended in an oil in water adjuvant. Group 2 was vaccinated twice intramuscularly with the bacterin vaccine, comprising the inactivated cells suspended in an oil in water adjuvant, and group 3 was left unvaccinated as challenge control. At 8 weeks of age the pigs were challenged intra-tracheally with a virulent culture of H. parasuis serotype 5.

During 10 days after challenge the pigs were observed daily for clinical signs of H. parasuis infection such as depression, locomotory problems and/or neurological signs and scored using a standard scoring system. Just before each vaccination and challenge, serum blood was collected for antibody determination. At regular times before and after challenge heparin blood was collected for re-isolation of challenge strain. Necropsy was performed on all animals that were culled before the scheduled day of necropsy as well as on all surviving animals.

Results

Before challenge, no abnormalities were observed and no intercurrent death occurred. Average survival time in days, average clinical scores, the number of animals that needed to be euthanized before the end of the study as well as the blood re-isolation results are indicated here below in table 2.

TABLE 2
Results of vaccination-challenge study

| | Average | Average | | # blood |
|---|---|---|---|---|
| | survival | clinical | # | culture |
| Group | time (days) | score | euthanised | positive |
| Subunit | 7.7 | 27.1 | 3/10 | 3/10 |
| Bacterin | 4.9 | 47.4 | 6/10 | 3/10 |
| Control | 2.0 | 71.0 | 9/10 | 6/10 |

### CONCLUSION

The results indicate that the recombinant subunit vaccine induced very good protection against H. parasuis challenge. Protection induced was superior to that induced by a bacterin vaccine.

1. (canceled)

2. The method of claim 9, wherein the protein antigen comprises an amino acid sequence that has at least 90% sequence identity with SEQ ID NO: 1, or an immunogenic fragment thereof.

3. The method of claim 2, wherein the protein antigen comprises an amino acid sequence that has at least 95% sequence identity with SEQ ID NO: 1, or an immunogenic fragment thereof.

4. The method of claim 3, wherein the protein antigen comprises the amino acid sequence of SEQ ID NO: 1, or an immunogenic fragment thereof.

5. The method of claim 9, wherein said administering of the vaccine protects the pig against an increased risk of mortality due to the infection with Haemophilus parasuis.

6. The method of claim 9, wherein said administering of the vaccine protects the pig against one or more clinical signs due to the infection with Haemophilus parasuis.

7. A vaccine to protect a pig against an infection with Haemophilus parasuis, the vaccine comprising a protein antigen comprising an amino acid sequence that has at least 69% sequence identity with SEQ ID NO: 1, or an immunogenic fragment thereof, and a pharmaceutically acceptable carrier.

8. (canceled)

9. A method to protect a pig against an infection with Haemophilus parasuis comprising administering a vaccine to the pig, wherein the vaccine comprises a protein antigen that comprises an amino acid sequence that has at least 69% sequence identity with SEQ ID NO: 1, or an immunogenic fragment thereof.

10. The vaccine of claim 7, wherein the protein antigen comprises an amino acid sequence that has at least 90% sequence identity with SEQ ID NO: 1, or an immunogenic fragment thereof, and a pharmaceutically acceptable carrier.

11. The vaccine of claim 10, wherein the protein antigen comprises an amino acid sequence that has at least 95% sequence identity with SEQ ID NO: 1, or an immunogenic fragment thereof, and a pharmaceutically acceptable carrier.

12. The vaccine of claim 7, wherein the protein antigen comprises the amino acid sequence of SEQ ID NO: 1, or an immunogenic fragment thereof, and a pharmaceutically acceptable carrier.
